(19) United States
(12) Patent Application Publication
(10) Pub. No.: US 2025/0276165 A1
(43) Pub. Date: Sep. 4, 2025

(54) VASCULAR ACCESS DEVICES, SYSTEMS, AND METHODS

(71) Applicant: Veris Health Inc, New York, NY (US)

(72) Inventors: James D. Mitchell, Walnut Creek, CA (US); Andrew A. Thoreson, Orono, MN (US); Lishan Aklog, Purchase, NY (US); Brian J. Deguzman, Paradise Valley, AZ (US); Stephen J. O'Neill, Waltham, MA (US)

(21) Appl. No.: 18/858,880

(22) PCT Filed: Apr. 28, 2023
(86) PCT No.: PCT/US2023/020479

§ 371 Date: Oct. 22, 2024

Related U.S. Application Data
(60) Provisional application No. 63/363,731, filed on Apr. 28, 2022.

(51) Int. Cl. A61M 39/02; A61M 39/04

(52) U.S. Cl. CPC A61M 39/0208; A61M 39/04; A61M 2205/3303; A61M 2205/3507; A61M 2210/12

(57) ABSTRACT

Vascular access devices and associated systems and methods are disclosed herein. According to some embodiments, the present technology includes a method of treatment comprising implanting a vascular access device through an incision at a first location and implanting an electronic device through the incision at a second location, the second location being different than the first location.

### CROSS-REFERENCE TO RELATED APPLICATIONS

The present application claims the benefit of priority to U.S. Provisional Application No. 63/363,731, filed Apr. 28, 2022.

### TECHNICAL FIELD

The present technology relates to implantable medical devices and associated systems and methods of use. Particular embodiments of the present technology are directed to vascular access devices, systems, and methods.

### BACKGROUND

Vascular access devices (e.g., vascular access ports) are minimally invasive, surgically implanted devices that provide relatively quick and easy access to a patient's central venous system for the purpose of administering intravenous medications, such as chemotherapeutic agents. Conventional vascular access devices are commonly used for patients requiring frequent, repeated intravenous administration of therapeutic agents or fluid, repeated blood draws, and/or for patients with difficult vascular access.

Vascular access devices such as vascular access ports typically include a reservoir attached to a catheter. The entire unit is placed completely within a patient's body using a minimally invasive surgical procedure. In most cases, the reservoir is placed in a small pocket created in the upper chest wall just inferior to the clavicle, and the catheter is inserted into the internal jugular vein or the subclavian vein with the tip resting in the superior vena cava or the right atrium. However, such vascular access devices can be placed in other parts of the body and/or with the catheter positioned in alternative sites as well. In conventional devices, the reservoir is typically bulky such that the overlying skin protrudes, allowing a clinician to use palpation to localize the device for access when it is to be used for a medication infusion or aspiration of blood for testing. A self-sealing cover (e.g., a thick silicone membrane) is disposed over and seals the reservoir, allowing for repeated access using a non-coring (e.g., Huber type) needle that is inserted through the skin and into the port. This access procedure establishes a system in which there is fluid communication between the needle, the vascular access device, the catheter, and the vascular space, thereby enabling infusion of medication or aspiration of blood via a transcutaneous needle.

Conventional vascular access devices are bulky by design to allow a clinician to localize the device by palpation. To be accurately accessed by a clinician, the vascular access device needs to be either visualized or palpated under the skin. Additionally, conventional vascular access ports have no electronic components and no internal power source. Accordingly, there is a need for improved vascular access devices.

### SUMMARY

The subject technology is illustrated, for example, according to various aspects described below, including with reference to FIGS. 1-9. Various examples of aspects of the subject technology are described as numbered clauses (1, 2, 3, etc.) for convenience. These are provided as examples and do not limit the subject technology.
1. A method of treatment, comprising:
implanting a vascular access device through an incision at a first location; and
implanting an electronic device through the incision at a second location, the second location being different than the first location.
2. A method of treatment, comprising:
securing a vascular access device to an electronic device via a coupler; and
implanting the vascular access device, the electronic device, and the coupler together through an incision.
3. A method of treatment, comprising:
implanting a vascular access device; and
implanting an electronic device after implanting the vascular access device; and
after implanting the electronic device, securing the vascular access device and the electronic device to a coupler.
4. A method of treatment, comprising:
implanting an electronic device; and
implanting a vascular access device after implanting the electronic device; and
after implanting the vascular access device, securing the vascular access device and the electronic device to a coupler.
5. A method of treatment, comprising:
securing a vascular access device to an electronic device by engaging a coupling portion of the vascular access device with a coupling portion of the electronic device; and
implanting the vascular access device and the electronic device together through an incision.
6. A method of treatment, comprising:
implanting a vascular access device; and
implanting an electronic device after implanting the vascular access device; and
after implanting the electronic device, securing the vascular access device to the electronic device by engaging a coupling portion of the vascular access device with a coupling portion of the electronic device.
7. A method of treatment, comprising:
implanting an electronic device; and
implanting a vascular access device after implanting the electronic device; and
after implanting the vascular access device, securing the vascular access device to the electronic device by engaging a coupling portion of the vascular access device with a coupling portion of the electronic device.
8. The method of any one of Clauses 1 to 5, wherein the vascular access device comprises:
a hub comprising a housing defining a reservoir configured to receive a fluid therein; and
a catheter comprising a proximal end portion configured to be secured to the hub, a distal end portion configured to be positioned within a body lumen of a patient, and a lumen extending therethrough, the lumen being in fluid communication with the reservoir.
9. The method of any one of Clauses 1 to 8, wherein the electronic device comprises:
a sensing element, the sensing element being configured to obtain data characterizing a physiological parameter of a patient.
10. The method of Clause 9, wherein the electronic device comprises:
a data communications module communicatively coupled to the sensing element and configured to transmit the data obtained by the sensing element to an external computing device.
11. The method of any one of Clauses 8 to 10, wherein the body lumen is a blood vessel.
12. The method of any one of Clauses 8 to 10, wherein the body lumen is a chamber or valve of the heart.
13. The method of any one of Clauses 8 to 10, wherein the body lumen is a peritoneal space, a renal pelvis or ureter, a bladder, a gallbladder, a stomach, a small bowel, a large bowel, a lung, a pleural space, or a pericardial space.
14. The method of any one of Clauses 2 to 4, wherein the coupler includes a first receiving portion configured to be secured to the vascular access device and a second receiving portion configured to be secured to the electronic device.
15. The method of Clause 1, wherein the first and second locations are within the same subcutaneous pocket.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic representation of a system for monitoring the health of a patient via an implanted medical system in accordance with the present technology.

FIG. 2 shows a vascular access device configured for use with the system of FIG. 1 and in accordance with several embodiments of the present technology.

FIG. 3 shows an electronic device configured for use with the system of FIG. 1 and in accordance with several embodiments of the present technology.

FIGS. 4A and 4B show a vascular access device and a coupler, respectively, configured for use with one another and in accordance with several embodiments of the present technology.

FIG. 4C shows the coupler of FIG. 4B coupled to a vascular access device and an electronic device in accordance with several embodiments of the present technology.

FIGS. 5A and 5B show a vascular access device and a coupler, respectively, configured for use with one another and in accordance with several embodiments of the present technology.

FIGS. 6A and 6B show a vascular access device and a coupler, respectively, configured for use with one another and in accordance with several embodiments of the present technology.

FIGS. 7A and 7B show a vascular access device and a coupler, respectively, configured for use with one another and in accordance with several embodiments of the present technology.

FIG. 8 shows a side cross-sectional view of a receiving portion of a coupler configured in accordance with several embodiments of the present technology.

FIG. 9 shows a coupler configured in accordance with several embodiments of the present technology.

FIGS. 10A-10D show a system configured in accordance with several embodiments of the present technology. FIGS. 10A-10C show the vascular access device and the electronic device coupled to the coupler. FIG. 10D is an isolated view of the coupler.

FIGS. 11A and 11B show a system in an assembled state and configured in accordance with several embodiments of the present technology.

FIG. 11C is an isolated view of the electronic device of the system shown in FIGS. 11A and 11B.

FIGS. 12A and 12B show a system in an assembled state and configured in accordance with several embodiments of the present technology.

FIG. 12C is an isolated view of the electronic device of the system shown in FIGS. 12A and 12B.

FIGS. 13A and 13B show a system in an assembled state and configured in accordance with several embodiments of the present technology.

FIG. 14 illustrates a system in an assembled state and configured in accordance with several embodiments of the present technology.

FIGS. 15A and 15B show a system in an assembled state and configured in accordance with several embodiments of the present technology.

FIGS. 16A and 16B show a system in an assembled state and configured in accordance with several embodiments of the present technology.

FIG. 16C is an isolated view of the electronic device of the system shown in FIGS. 16A and 16B.

FIGS. 17A-17C show a system in various configurations in accordance with several embodiments of the present technology.

FIG. 18 illustrates a system in as assembled state and configured in accordance with several embodiments of the present technology.

FIGS. 19A and 19B show a system in an assembly state and configured in accordance with several embodiments of the present technology.

### DETAILED DESCRIPTION

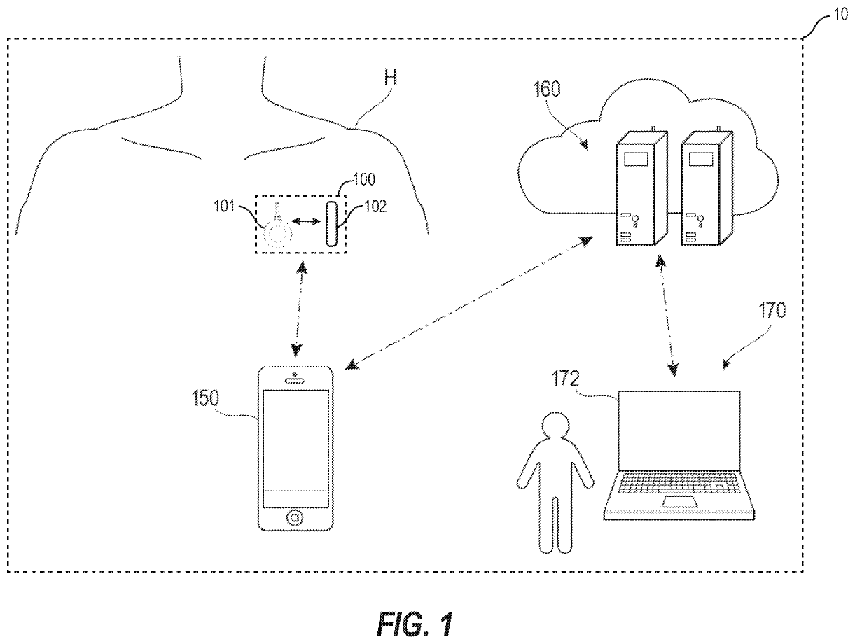

The present technology relates to implantable medical devices such as vascular access devices and associated systems and methods of use. Specific details of several embodiments of the technology are described below with reference to FIGS. 1-9.

The devices and systems of the present technology may be equipped with electronic components that provide a platform for remote monitoring of the device and/or patient. For example, several of the systems disclosed herein include an implantable vascular access device and a separate, implantable electronic device. The electronic device can include a sensing element that is configured to obtain patient physiological data while the electronic device is implanted within the patient, and determine one or more physiological parameters based on the data. The system may determine certain physiological parameters, for example, that indicate one or more symptoms of a medical condition that requires immediate medical attention or hospitalization. Such physiological parameters can include those related to temperature, patient movement/activity level, heart rate, respiratory rate, blood oxygen saturation, and/or other suitable parameters described herein. The vascular access device and the electronic device may be implanted at the same time and through the same incision, which is convenient for the physician and procedurally cost effective. In some embodiments the vascular access device and electronic device are coupled to one another prior to implantation (i.e., ex vivo). According to some embodiments, the vascular access device and electronic device are coupled to one another in vivo.

According to various embodiments of the present technology, the vascular access device comprises a fluid reservoir and a catheter configured to be fluidically coupled to the reservoir. The vascular access device is configured to be implanted in a subcutaneous pocket at an accessible location (such as the chest), and the catheter can extend from the reservoir to a remote vascular location to provide convenient, repeatable access to the patient's venous or arterial system. In some embodiments, the catheter can provide access to a body cavity or hollow organ such as the peritoneal space, the renal pelvis or ureter, the bladder, the gallbladder, the stomach, the small bowel, the large bowel, the lung, the pleural space, or the pericardial space.

FIG. 1 is a schematic representation of a system 10 for remote monitoring and vascular access. The system 10 comprises a modular implantable system 100 in accordance with the present technology. The implantable system 100 is configured to be implanted within a human patient H at an accessible location, such as at a subcutaneous location along an upper region of the patient's chest. For example, according to several embodiments, the implantable system 100 can be implanted in a subcutaneous pocket created in the patient's upper chest wall, just inferior to the clavicle. As shown in FIG. 1, the implantable system 100 may include a vascular access device 101 and an electronic device 102. The vascular access device 101 can comprise a reservoir configured to receive a therapeutic agent for intravascular administration, and a catheter extending from the reservoir and configured to be inserted into the vasculature at a remote location. The vascular access device 101 and the electronic device 102 can be implanted in the same or different locations and at the same time or at different times, as discussed in greater detail below.

The electronic device 102 can include electronic components that are configured to obtain data characterizing a physiological parameter of the patient and communicate that data to the system 10. In some embodiments, the electronic device 102 includes a sensing element that is configured to obtain physiological measurements that are used by the system 10 to determine one or more physiological parameters indicative of the patient's health. As used herein, “sensing element” can refer to a single sensor or multiple sensors. The system 10 may detect a medical condition or associated symptom(s) based on the physiological parameter(s) and, optionally, provide an indication of the detected condition to the patient, caregiver, and/or medical care team.

As shown schematically in FIG. 1, the electronic device 102 may be configured to communicate wirelessly with a local computing device 150, which can be, for example, a smart device (e.g., a smartphone, a tablet, or other handheld device having a processor and memory), a special-purpose interrogation device, or other suitable device. Communication between the implantable system 100 and the local computing device 150 can be mediated by, for example, near-field communication (NFC), infrared wireless, Bluetooth, ZigBee, Wi-Fi, inductive coupling, capacitive coupling, or any other suitable wireless communication link. The implantable system 100 may transmit data including, for example, measurements obtained via the sensing element characterizing physiological parameters of the patient, patient medical records, device performance metrics (e.g., battery level, error logs, etc.), or any other such data obtained by or stored by the implantable system 100. In some embodiments, the transmitted data is encrypted or otherwise obfuscated to maintain security during transmission to the local computing device 150. The local computing device 150 may also provide instructions to the electronic device 102, for example to obtain certain physiological measurements via the sensing element, to emit a localization signal, or to perform other functions. In some embodiments, the local computing device 150 may be configured to wirelessly recharge a battery of the implantable system 100, for example via inductive charging.

The system 10 may further include first remote computing device(s) 160 (or server(s)), and the local computing device 150 may in turn be in communication with first remote computing device(s) 160 over a wired or wireless communications link (e.g., the Internet, public and private intranet, a local or extended Wi-Fi network, cell towers, the plain old telephone system (POTS), etc.). The first remote computing device(s) 160 may include one or more own processor(s) and memory. The memory may be a tangible, non-transitory computer-readable medium configured to store instructions executable by the processor(s). The memory may also be configured to function as a remote database, i.e., the memory may be configured to permanently or temporarily store data received from the local computing device 150 (such as one or more physiological measurements or parameters and/or other patient information).

In some embodiments, the first remote computing device(s) 160 can additionally or alternatively include, for example, server computers associated with a hospital, a medical provider, medical records database, insurance company, or other entity charged with securely storing patient data and/or device data. At a remote location 170 (e.g., a hospital, clinic, insurance office, medical records database, operator's home, etc.), an operator may access the data via a second remote computing device 172, which can be, for example a personal computer, smart device (e.g., a smartphone, a tablet, or other handheld device having a processor and memory), or other suitable device. The operator may access the data, for example, via a web-based application. In some embodiments, the obfuscated data provided by the implantable system 100 can be de-obfuscated (e.g., unencrypted) at the remote location 170.

In some embodiments, the electronic device 102 may communicate with remote computing devices 160 and/or 172 without the intermediation of the local computing device 150. For example, the electronic device 102 may be connected via Wi-Fi or other wireless communications link to a network such as the Internet. In other embodiments, the electronic device 102 may be in communication only with the local computing device 150, which in turn is in communication with remote computing devices 160 and/or 172.

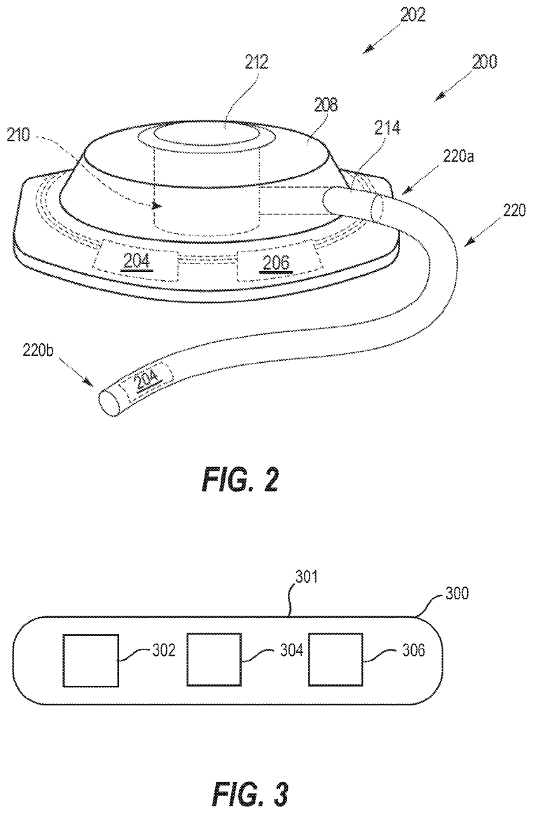

FIG. 2 shows an example of a vascular access device 200 (or “device 200”) configured for use with the systems 10 and implantable systems 100 of the present technology. As shown in FIG. 2, the device 200 comprises a hub 202 and a catheter 220 configured to be permanently or detachably coupled to the hub 202. The hub 202 may comprise a housing 208, a fluid reservoir 210 contained within the housing 208, and a septum 212 adjacent the reservoir 210 that is configured to receive a needle therethrough for delivery of a fluid (such as a therapeutic or diagnostic agent) to the reservoir 210. The housing 208 may be made of a biocompatible plastic, metal, ceramic, medical grade silicone, or other material that provides sufficient rigidity and strength to prevent inadvertent needle puncture through the housing 208. The septum 212 can be, for example, a self-sealing membrane made of silicone or other deformable, self-sealing, biocompatible material.

The catheter 220 can be configured to permanently or detachably couple to the hub 202 to be placed into fluid communication with the reservoir 210. For example, as shown in FIG. 2, the catheter 220 can have a proximal end portion 220a configured to mate with an outlet port 214 of the hub 202 (e.g., via a barb connector or other suitable mechanical connection) and a distal end portion 220b configured to be positioned within a blood vessel or a heart of a patient. The device 200 and/or implantable system 100 can comprise a single catheter 220 (as shown in FIG. 2) or multiple catheters 220. Moreover, the catheter 220 can define a single lumen or multiple lumens.

FIG. 3 schematically depicts an electronic device 300 (or “device 300”) configured for use with the systems 10 and implantable systems 100 of the present technology. The electronic device 300 is configured to obtain measurements indicative of a health of the patient as well as communicate with the system 100. The electronic device 300 may include a sensing element 302, a controller 304, and a communications unit 306, all carried by a housing 301. The housing 301, for example, can comprise a hermetically sealed enclosure. In some embodiments, the sensing element 302, controller 304, and/or communications unit 306 are carried by a printed circuit board (PCB). As discussed in greater detail below, the sensing element 302 can be configured to obtain data characterizing an identity parameter of the patient, a physiological parameter of the patient. As used herein, the term “sensing element” may refer to a single sensor or a plurality of discrete, separate sensors. Although one sensing element 302 is illustrated for clarity in FIG. 3, in various embodiments, the device 300 may include more than two sensing elements 302 (e.g., two sensing elements, three sensing elements, four sensing elements, etc.).

As shown in FIG. 3, in some embodiments the device 300 includes a power source 308, such as a battery. In several of such embodiments, the device 300 can include an antenna (e.g., a coil) (not shown) that is configured to be inductively coupled to an external antenna (e.g., a coil). The electrical energy generated in the device antenna by the external antenna can be used to recharge and/or power the power source 308. In certain embodiments, the device 300 does not include a power source 308 and only includes the device antenna. In such embodiments, the device 300 may only be powered when in the presence of the external antenna. In any case, the external antenna can be, for example, an interrogation device (e.g., local device 150 or another suitable device) or other device.

As shown in FIG. 3, the electronic device 300 can have an elongated shape with a slim profile for improving patient comfort. In other embodiments, the electronic device 300 can have other shapes and configurations.

The controller 304 may include one or more processors, software components, and/or memory (not shown). In some examples, the one or more processors include one or more computing components configured to process measurements received from the sensing element 302 according to instructions stored in the memory. The memory may be a tangible, non-transitory computer-readable medium configured to store instructions executable by the one or more processors. For instance, the memory may be data storage that can be loaded with one or more of the software components executable by the one or more processors to achieve certain functions. In some examples, the functions may involve causing the sensing element 302 to obtain one or more measurements, such as data characterizing a physiological parameter of the patient. In another example, the functions may involve processing the data to determine one or more parameters and/or provide an indication to the patient and/or clinician of a health of the patient. For example, the functions may involve providing an indication to the patient and/or clinician of one or more symptoms or medical conditions associated with determined physiological parameters.

The communications unit 306 can be configured to securely transmit data between the device 300 and external computing devices (e.g., local computing device 150, remote computing devices 160 and 172, etc.). In some embodiments, the controller 304 includes a localization unit configured to emit a localization signal (e.g., lights that transilluminate a patient's skin, vibration, a magnetic field, etc.) to aid a clinician in localizing the device 300 when implanted within a patient.

The system 10 may be configured to continuously and/or periodically obtain measurements via the sensing element 302.

In some embodiments, the sensing element 302 is built into the housing 301 such that only a portion of the sensing element 302 is exposed to the local physiological environment when the device 300 is implanted. For example, the sensing element 302 may comprise one or more electrodes having an external portion positioned at an exterior surface of the housing 301 and an internal portion positioned within the housing 301 and, optionally, wired to the controller 304. In some embodiments the sensing element 302 may be completely contained within the housing 301. For example, the sensing element 302 may comprise one or more pulse oximeters enclosed by the housing 301 and positioned adjacent a window in the housing 301 through which light emitted from the pulse oximeter may pass to an external location, and back through which light reflected from the external location may pass for detection by a photodiode of the pulse oximeter. In such embodiments the window may be, for example, a sapphire window that is brazed into place within an exterior wall of the housing 301.

The sensing element 302 may comprise at least one sensor completely enclosed by the housing 301 and at least one sensor that is partially or completely positioned at an external location, whether directly on the housing 301 or separated from the housing 301 (but still physically coupled to the housing 301 via a wired connection, for example).

In some embodiments, the controller 304 processes at least some of the measurements to determine one or more parameters, and then transmits those parameters to one or more of the local computing device 150, remote computing devices 160, and/or remote computing devices 172 (with or without the underlying data). In some examples, the controller 304 may only partially process at least some of the measurements before transmitting the data to the local computing device 150, remote computing devices 160, and/or remote computing devices 172. In such embodiments, the controller 304 may further process the received data to determine one or more parameters. The local computing device 150, the remote computing devices 160, and/or the remote computing devices 172 may also process some or all of the measurements obtained by the sensing element 302 and/or parameters determined by the sensing element 302 and/or the controller 304.

In some embodiments, the sensing element(s) 302 and/or controller 304 may be configured to detect, identify, monitor, and/or communicate information by electromagnetic, acoustic, motion, optical, thermal, or biochemical sensing elements or means. The sensing element(s) 302 may include, for example, one or more temperature sensing elements (e.g., one or more thermocouples, one or more digital temperature sensors, one or more thermistors or other type of resistance temperature detector, etc.), one or more impedance sensing elements (e.g., one or more electrodes), one or more pressure sensing elements, one or more optical sensing elements, one or more flow sensing elements (e.g., a Doppler velocity sensing element, an ultrasonic flow meter, etc.), one or more ultrasonic sensing elements, one or more photoplethysmography (PPG) sensing elements (e.g., pulse oximeters, etc.), one or more chemical sensing elements, one or more movement sensing elements (e.g., one or more accelerometers), one or more pH sensing elements, an electrocardiogram (“ECG” or “EKG”) unit, one or more electrochemical sensing elements, one or more hemodynamic sensing elements, and/or other suitable sensing devices.

The sensing element 302 may comprise one or more electromagnetic sensing elements configured to measure and/or detect, for example, impedance, voltage, current, or magnetic field sensing capability with a wire, wires, wire bundle, magnetic node, and/or array of nodes. The sensing element 302 may comprise one or more acoustic sensing elements configured to measure and/or detect, for example, sound frequency, within human auditory range or below or above frequencies of human auditory range, beat or pulse pattern, tonal pitch melody, and/or song. The sensing element 302 may comprise one or more motion sensing elements configured to measure and/or detect, for example, vibration, movement pulse, pattern or rhythm of movement, intensity of movement, and/or speed of movement. Motion communication may occur by a recognizable response to a signal. This response may be by vibration, pulse, movement pattern, direction, acceleration, or rate of movement. Motion communication may also be by lack of response, in which case a physical signal, vibration, or bump to the environment yields a motion response in the surrounding tissue that can be distinguished from the motion response of the sensing element 302. Motion communication may also be by characteristic input signal and responding resonance. The sensing element 302 may comprise one or more optical sensing elements which may include, for example, illuminating light wavelength, light intensity, on/off light pulse frequency, on/off light pulse pattern, passive glow or active glow when illuminated with special light such as UV or “black light”, or display of recognizable shapes or characters. It also includes characterization by spectroscopy, interferometry, response to infrared illumination, and/or optical coherence tomography. The sensing element 302 may comprise one or more thermal sensing elements configured to measure and/or detect, for example, the temperature of the device 300 relative to surrounding environment, the temperature of the device 300 (or portion thereof), the temperature of the environment surrounding the device 300 and/or sensing element 302, or differential rate of the device temperature change relative to surroundings when the device environment is heated or cooled by external means. The sensing element 302 may comprise one or more biochemical devices which may include, for example, the use of a catheter, a tubule, wicking paper, or wicking fiber to enable micro-fluidic transport of bodily fluid for sensing of protein, RNA, DNA, antigen, and/or virus with a micro-array chip.

In some aspects of the technology, the controller 304 and/or sensing element 302 may be configured to detect and/or measure the concentration of blood constituents, such as sodium, potassium, chloride, bicarbonate, creatinine, blood urea nitrogen, calcium, magnesium, and phosphorus. The system 10 and/or the sensing element 302 may be configured to evaluate liver function (e.g., by evaluation and/or detection of AST, ALT, alkaline phosphatase, gamma glutamyl transferase, troponin, etc.), heart function (e.g., by evaluation and/or detection of troponin, brain natriuretic peptide (BNP)), coagulation (e.g., via determination of prothrombin time (PT), partial thromboplastin time (PTT), and international normalized ratio (INR)), and/or blood counts (e.g., hemoglobin or hematocrit, white blood cell levels with differential, and platelets). In some embodiments, the system 10 and/or the sensing element 302 may be configured to detect and/or measure circulating tumor cells, circulating tumor DNA, circulating RNA, multigene sequencing of germ line or tumor DNA, markers of inflammation such as cytokines, C reactive protein, erythrocyte sedimentation rate, tumor markers (PSA, beta-HCG, AFP, LDH, CA 125, CA 19-9, CEA, etc.), seizure activity (procalcitonin), and others.

As previously mentioned, the system 10 may determine one or more physiological parameters based on data obtained by the sensing element 302 and/or one or more other physiological parameter(s). For example, the system 10 may be configured to determine physiological parameters such as heart rate, temperature, blood pressure (e.g., systolic blood pressure, diastolic blood pressure, mean arterial blood pressure), cardiac output, ejection fraction, pulmonary artery pressure, pulmonary capillary wedge pressure, left atrial pressure, blood flow rate, blood velocity, pulse wave speed, volumetric flow rate, reflected pressure wave amplitude, augmentation index, flow reserve, resistance reserve, resistive index, capacitance reserve, hematocrit, heart rhythm, electrocardiogram (ECG) tracings, body fat percentage, activity level, body movement, falls, gait analysis, seizure activity, blood glucose levels, drug/medication levels, blood gas constituents and blood gas levels (e.g., oxygen, carbon dioxide, etc.), lactate levels, hormone levels (such as cortisol, thyroid hormone (T4, T3, free T4, free T3), TSH, ACTH, parathyroid hormone), medication concentration in the blood, pharmacokinetic and pharmacodynamic data, and/or any correlates and/or derivatives of the foregoing measurements and parameters (e.g., raw data values, including voltages and/or other directly measured values). In some embodiments, data obtained by the sensing element 302 can be utilized or characterized as a physiological parameter without any additional processing by the system 10.

The system 10 may also determine and/or monitor derivatives of any of the foregoing parameters (e.g., physiological parameters, device performance parameters, treatment parameters, identity parameters, etc.), such as a rate of change of a particular parameter, a change in a particular parameter over a particular time frame, etc. As but a few examples, the system 10 may be configured to determine a temperature over a specified time, a maximum temperature, a maximum average temperature, a minimum temperature, a temperature at a predetermined or calculated time relative to a predetermined or calculated temperature, an average temperature over a specified time, a maximum blood flow, a minimum blood flow, a blood flow at a predetermined or calculated time relative to a predetermined or calculated blood flow, an average blood flow over time, a maximum impedance, a minimum impedance, an impedance at a predetermined or calculated time relative to a predetermined or calculated impedance, a change in impedance over a specified time, a change in impedance relative to a change in temperature over a specified time, a change in heart rate over time, a change in respiratory rate over time, activity level over a specified time and/or at a specified time of day, and other suitable derivatives.

Data may be obtained continuously or periodically at one or more predetermined times, ranges of times, calculated times, and/or times when or relative to when a measured event occurs. Likewise, parameters (physiological or otherwise) may be determined continuously or periodically at one or more predetermined times, ranges of times, calculated times, and/or times when or relative to when a measured event occurs.

Based on the determined parameters, the system 10 of the present technology can be configured to provide an indication of the patient's health, the performance and/or health of device, and/or the status of a treatment to the patient and/or a clinician. For example, the controller may compare one or more physiological parameters to a predetermined threshold or range and, based on the comparison, provide an indication of the patient's health. If the determined physiological parameter(s) is above or below the predetermined threshold or outside of the predetermined range, the system 10 may provide an indication that the patient is at risk of, or has already developed, a medical condition characterized by symptoms associated with the determined physiological parameters. As used herein, a “predetermined range” refers to a set range of values, and “outside of a/the predetermined range” refers to (a) a measured or calculated range of values that only partially overlap the predetermined range or do not overlap any portion of a predetermined range of values. As used herein, a “predetermined threshold” refers to a single value or range of values, and a parameter that is “outside” of “a predetermined threshold” refers to a situation where the parameter is (a) a measured or calculated value that exceeds or fails to meet a predetermined value, (b) a measured or calculated value that falls outside of a predetermined range of values, (c) a measured or calculated range of values that only partially overlaps a predetermined range of values or does not overlap any portion of a predetermined range of values, or (d) a measured or calculated range of values where none of the values overlap with a predetermined value.

Predetermined parameter thresholds and/or ranges can be empirically determined to create a look-up table. Look-up table values can be empirically determined, for example, based on clinical studies and/or known healthy or normal values or ranges of values. The predetermined threshold may additionally or alternatively be based on a particular patient's baseline physiological parameters, a particular device's baseline performance parameters, etc. In some embodiments, the system 10 can be configured to determine the predetermined threshold and/or range based on data collected by the device to determine a patient's normal or baseline parameter or range of parameters. In some cases, a patient's baseline parameter or range of parameters may differ from known normal parameters/ranges of parameters. For example, a patient with anemia may have a lower baseline hemoglobin level, hematocrit, or red blood cell count than a non-anemic patient. Accordingly, a predetermined threshold for hemoglobin that indicates that the patient's hemoglobin is abnormal and/or indicative of a medical condition may be lower for the anemic patient than for a non-anemic patient.

In some embodiments, the system 10 may be configured to detect a pattern of measurements (of a single parameter or a combination of parameters) indicative of a health condition. In such embodiments, the individual measurements may not fall outside of a given “normal” range yet, when considered together, can still indicate a change in health status. For example, the controller may be configured to identify patterns of change in temperature, heart rate, and activity that are associated with infection, even if all three are still within a “normal” range.

Medical conditions detected and/or indicated by the system 10 may include, for example, sepsis, pulmonary embolism, metastatic spinal cord compression, anemia, dehydration/volume depletion, vomiting, pneumonia, congestive heart failure, performance status, arrythmia, neutropenic fever, acute myocardial infarction, pain, opioid toxicity, nicotine or other drug addiction or dependency, hyperglycemic/diabetic ketoacidosis, hypoglycemia, hyperkalemia, hypercalcemia, hyponatremia, one or more brain metastases, superior vena cava syndrome, gastrointestinal hemorrhage, immunotherapy-induced or radiation pneumonitis, immunotherapy-induced colitis, diarrhea, cerebrovascular accident, stroke, pathological fracture, hemoptysis, hematemesis, medication-induced QT prolongation, heart block, tumor lysis syndrome, sickle cell anemia crisis, gastroparesis/cyclic vomiting syndrome, hemophilia, cystic fibrosis, chronic pain, volume overload, hyperuricemia, and/or seizure. Any of the systems and/or devices disclosed herein may be used to monitor a patient for any of the foregoing medical conditions.

The system 10 can be configured to provide notifications to a patient and/or a clinician. For example, the device 300 and/or an external computing device (e.g., a smartphone, a PC, etc.) can be configured to provide a notification to the patient if a battery carried by the electronic device 102 is low and needs to be recharged. The system 10 may be configured to provide a notification to a patient and/or clinician if it determines that the device is occluded, overheating, has lost wireless communication, is infected or colonized, or is otherwise malfunctioning. In some embodiments, the electronic device 102 includes a notification unit configured to provide notifications to the patient without the need for an external computing device. The notification unit can include a speaker, a light, a vibration element, or another means for providing audible, visual, haptic, and/or tactile notifications to the patient. As an example, the notification unit can comprise a vibration element configured to vibrate if the patient's blood glucose has fallen outside of predetermined healthy or normal range to indicate to the patient that intervention should be taken to regulate their blood glucose.

In some embodiments, one or more parameters of a notification provided by a notification unit can be based on a type of information to be communicated to a patient. For example, a high frequency, high amplitude vibration can communicate to the patient that their blood pressure is higher than a predetermined threshold, while a low frequency, low amplitude vibration can communicate to the patient that their blood pressure is lower than a predetermined threshold. Further, various notification modalities and/or parameters can be used alone or in combination to communicate specific information to the patient. For example, the notification unit can include an LED light and a speaker. If the system 10 determines that the patient is experiencing ventricular fibrillation, the LED light can emit red light and the speaker can emit an audible notification instructing listeners to call an ambulance. In some embodiments, the LED light can emit a light of a specific color to indicate that the electronic device 102 needs to be recharged.

According to various embodiments of the present technology, the device 300 can comprise a voice recognition unit configured to obtain audio data. The voice recognition unit can include a microphone, such as any of the microphones described herein, and a controller communicatively coupled to the microphone. In some embodiments, the voice recognition unit or one or more portions thereof is communicatively coupled to the sensing element 302 or another electronic component carried by the device 300. In some embodiments, the voice recognition unit comprises a microphone communicatively coupled to a separate controller carried by device 300 and/or an external controller.

The voice recognition unit can be configured to obtain audio data and, in some embodiments, transmit the audio data to the controller 304. In some embodiments, the controller 304 can be configured to cause the device 300 to perform a function or action, based at least in part on the audio data. For example, a person (e.g., a patient, a clinician, etc.) can provide an audible instruction for the device 300 to obtain data characterizing a temperature of the person. Providing the audible instruction can include saying out loud “Device, take the patient's temperature.” The voice recognition unit can obtain audio data characterizing the sound waves produced by the person providing the audible instruction. The audio data can be transmitted to a controller, which can process the audio data and, based on the audio data, cause the sensing element 302 carried by the device 300 to obtain data characterizing a temperature of the patient. In some embodiments, the controller can process the temperature data and/or cause the notification unit to provide a notification communicating the patient's temperature. In another example, a person can provide an audible instruction for the device 300 to turn on, wake up, exit a lower-power mode, or otherwise activate.

According to some methods of use, the vascular access device 200 can be implanted through an incision at a first location, and the electronic device 300 can be implanted through the incision at a second location that is different than the first location. For example, an incision can be made in the upper chest region to create a subcutaneous pocket. The vascular access device 200 can be delivered through the incision to the subcutaneous pocket, and then the electronic device 300 can be separately delivered through the same incision to the subcutaneous pocket. Alternatively, the electronic device 300 can be delivered through the incision to the subcutaneous pocket, and then the vascular access device 200 can be separately delivered through the same incision to the subcutaneous pocket.

In some embodiments, the vascular access device 200 can be free-floating relative to the electronic device 300. Alternatively, the vascular access device 200 and electronic device 300 can be mechanically coupled to one another, for example by joining them together directly or via a coupler. According to some methods of use, the vascular access device 200 and electronic device 300 are coupled to one another prior to implantation (i.e., ex vivo). According to some methods of use, the vascular access device 200 and the electronic device 300 are coupled to one another in vivo.

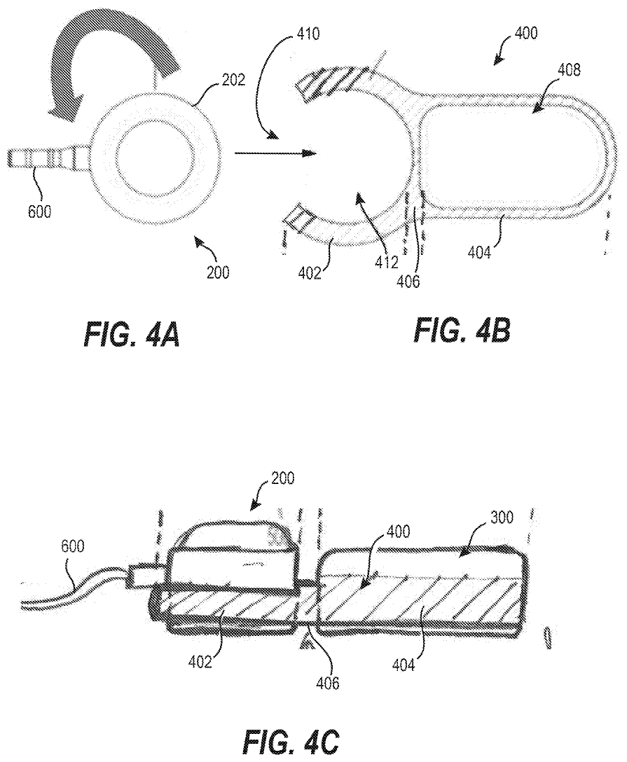

FIGS. 4A-9B depict various coupler embodiments configured in accordance with the present technology. The couplers shown in FIGS. 4A-9 can be used with any of the vascular access devices 200 and electronic devices 300 disclosed herein.

FIGS. 4A, 4B, and 4C, for example, show a coupler 400 and associated implantable system. FIG. 4B shows the coupler 400 prior to engagement with the vascular access device 200 and electronic device 300. The coupler 400 can comprise a flexible housing having a first receiving portion 402 configured to receive the vascular access device 200 and a second receiving portion 404 configured to receive the electronic device 300. The housing may further include an intermediate portion 406 between the first and second receiving portions 402, 404. The intermediate portion 406 can be sufficiently flexible to allow some movement between the first and second receiving portions 402, 404. For example, the housing can be configured to flex and/or bend at the intermediate portion 406 so that the system 100 can better conform to the curvature of the patient's chest (or other implantation location), thereby improving patient comfort. In some embodiments, the intermediate portion 406 can be relatively thin to encourage preferential bending at the intermediate portion 406. Alternatively, the intermediate portion 406 may be rigid such that the devices 200 and 300 are held in a desired spatial relationship. In some embodiments, the intermediate portion 406 can comprise a material that is sufficiently soft and/or thin such that the coupler 400 can be easily cut in vivo, at the intermediate portion 406, to separate the devices 200 and 300. Such a detachable arrangement may be beneficial should the need arise to remove only one of the devices 200 and 300 from the system.

The first receiving portion 402 can comprise a sidewall 403 that defines an opening 412 and at least partially outlines a shape complementary to that of the vascular access device 200. For example, in some embodiments the vascular access device 200 has a circular shape, and the sidewall of the first receiving portion 402 circumscribes a portion of the circular shape. The first receiving portion 402 can have a resting diameter that is slightly less than the diameter of the vascular access device 200, and the sidewall can be configured to stretch to accommodate the larger vascular access device 200. The sidewall can be sufficiently elastic such that, in its deformed state, it exerts enough compressive force on the vascular access device 200 to secure the vascular access device 200 thereto. In some embodiments, the first receiving portion 402 is configured to receive and be secured to the vascular access device 200 in a snap fit arrangement.

In some embodiments, the first receiving portion 402 can have a gap 410 in the sidewall. As indicated by FIGS. 4A and 4B together, and as shown in the assembled side view of FIG. 4C, the hub 202 of the vascular access device 200 can be configured to be positioned in the first receiving portion 402 such that the catheter 600 extends through the gap 410 in the sidewall. The gap 410 can have an arc length that allows the vascular access device 200 to rotate within the first receiving portion 402 so that the catheter 600 can be positioned at a variety of angles to accommodate the unique anatomical environments presented by different patients. In some embodiments, the arc length allows the vascular access device 200 to rotate between 10 and 180 degrees.

The second receiving portion 404 can comprise a sidewall 405 that defines an opening 408 and at least partially outlines a shape complementary to that of the electronic device 300. For example, in some embodiments the electronic device 300 has an elongated shape, and the sidewall of the second receiving portion 404 circumscribes the entire elongated shape. The second receiving portion 404 can have a resting diameter that is slightly less than the diameter of the electronic device 300, and the sidewall can be configured to stretch to accommodate the larger electronic device 300. The sidewall can be sufficiently elastic such that, in its deformed state, it exerts enough compressive force on the electronic device 300 to secure the electronic device 300 thereto. In some embodiments, the second receiving portion 404 is configured to receive and be secured to the electronic device 300 in a snap fit arrangement.

In some embodiments, for example as shown in FIGS. 4B and 4C, the first and second receiving portions 402, 404 only comprise a respective sidewall 403, 405 and do not include any portion that extends across all or a portion of the top or bottom sides of respective openings 412, 408. As such, when the vascular access device 200 and electronic device 300 are positioned in the first and second receiving portions 402, 404, respectively, the top and bottom surfaces of the vascular access device 200 and electronic device 300 are exposed. In some embodiments, the first receiving portion 402 can comprise a bottom support (not shown) that covers all or a portion of the opening 412 at the bottom side of the sidewall 403. The top side of the first receiving portion 402 may remain open, however, so that the reservoir can still be accessed by a needle. Additionally or alternatively, the second receiving portion 404 can comprise a bottom support (not shown) that covers all or a portion of the opening 408 at the bottom side of the sidewall 405. Additionally or alternatively, the second receiving portion 404 can comprise a top support (not shown) that covers all or a portion of the opening 408 at the top side of the sidewall 405. In those embodiments in which the second receiving portion 404 has both top and bottom supports, the sidewall 405 can have a gap (similar to gap 410) to allow the device 300 to be inserted into the opening 408. In any of the foregoing embodiments, any portion of the first and/or second receiving portions 402, 404 (e.g., the sidewall, the top support, the bottom support, etc.) can have apertures extending therethrough that enable contact between electrodes and/or other sensing elements of the respective device 200, 300 and adjacent body tissue.

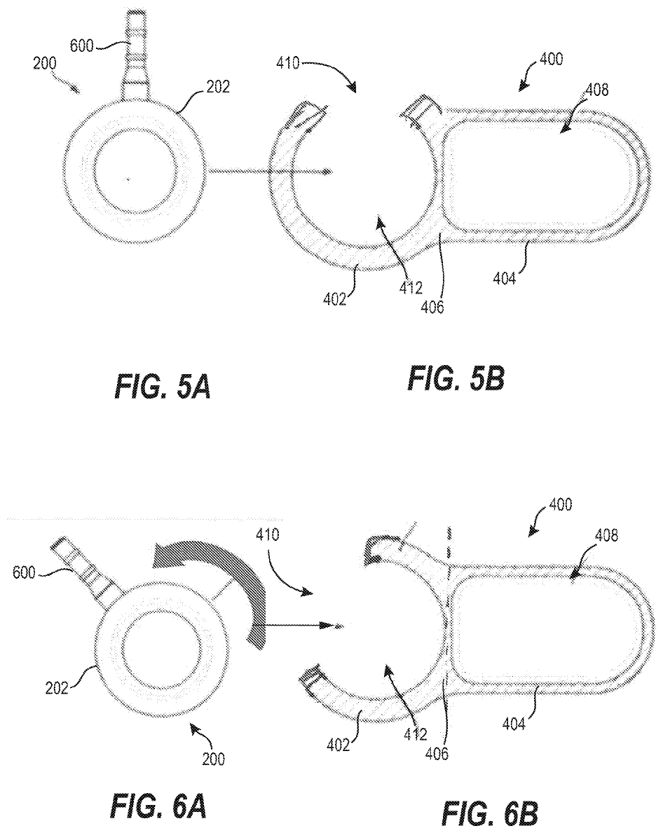

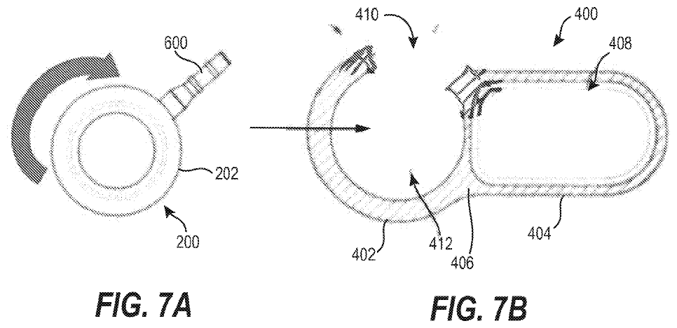

As shown in FIG. 4B, the gap 410 can be positioned along a side of the first receiving portion 402 that is laterally opposite of the intermediate portion 406 and/or second receiving portion 404. As such, the catheter 600 is configured to extend laterally away from the intermediate portion 406 and/or second receiving portion 404. In other embodiments, the gap 410 can be positioned elsewhere along the first receiving portion 402. For example, as shown in FIGS. 5A and 5B, the gap 410 can be positioned along the first receiving portion 402 such that, when the vascular access device 200 is positioned within the first receiving portion 402, the catheter 600 extends substantially perpendicular to the longitudinal axis of the second receiving portion 404 (plus or minus 5-10 degrees). As shown in FIGS. 6A and 6B, in some embodiments the gap 410 is positioned along the first receiving portion 402 such that, when the vascular access device 200 is positioned within the first receiving portion 402, the catheter 600 extends laterally away from the second receiving portion 404 and forms an angle of about 135 degrees with the longitudinal axis of the second receiving portion 404 (plus or minus 5-10 degrees). As shown in FIGS. 7A and 7B, in some embodiments the gap 410 is positioned along the first receiving portion 402 such that, when the vascular access device 200 is positioned within the first receiving portion 402, the catheter 600 extends laterally towards the second receiving portion 404 and forms an angle of about 45 degrees with the longitudinal axis of the second receiving portion 404 (plus or minus 5-10 degrees).

For any of the foregoing embodiments, it will be appreciated that the gap in the first receiving portion can be positioned at any location along the circumference and/or curved boundary defining the first receiving portion. Likewise, the gap can have any width that is sufficient to receive the vascular access device 200 therethrough.

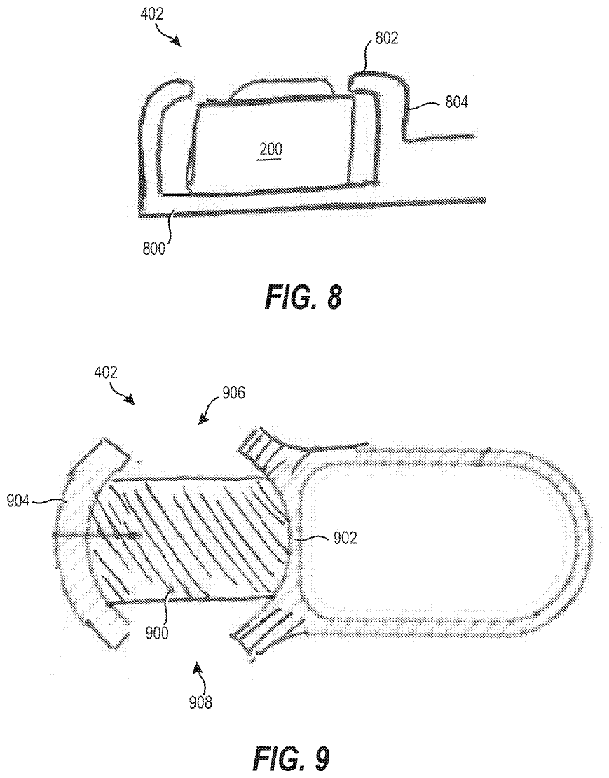

As shown in FIGS. 8 and 9, in some embodiments the housing of the coupler 400 can have a bottom support 800 and a lip 802 that extends radially inwardly from a top portion of the sidewall 804 defining the first receiving portion 402. The lip 802 can be configured to extend over a portion of the vascular access device 200 positioned therein, thereby limiting and/or preventing vertical movement of the vascular access device 200 relative to the coupler 400. The lip 802 can extend around the entire perimeter of the sidewall 804 of the first receiving portion 402, or may extend around less than the entire perimeter of the sidewall 804. To secure the vascular access device 200 to the first receiving portion 402, the vascular access device 200 can be introduced through a top opening in the first receiving portion 402, thereby initially forcing the lip 802 to bend downwardly. Once the vascular access device 200 is positioned against the bottom support 800, the lip 802 can be free to return to its resting position, which extends over a portion of the vascular access device 200. Additionally or alternatively, the second receiving portion 404 can have a bottom surface and a lip extending radially inwardly along all or a portion of its sidewall.

As shown in FIG. 9, in some embodiments the first receiving portion 402 can comprise a bottom support 900 that spans less than the entire area defined by the sidewalls of the first receiving portion 402. In such embodiments, the first receiving portion 402 can have a first sidewall 902 closest to the intermediate portion 406 and a second sidewall 904 laterally opposite of the first sidewall 902 and connected to the first sidewall 902 via the bottom support 900. The first receiving portion 402 can further include first and second gaps 906, 908 between the first and second sidewalls 902, 904. The end portions of the sidewalls 902, 904 (e.g., closest to the gaps 906, 908) can be configured to bend in a radial direction to facilitate insertion of the vascular access device 200 through the side gaps 906, 908. For example, when inserting the vascular access device 200, the ends may bend radially inwardly, then spring back to their resting positions when the vascular access device 200 is fully inserted. Likewise, when the vascular access device 200 is removed through one of the side gaps 906, 908, the end portions can bend radially outwardly to allow passage of the vascular access device 200, then return to their resting positions when the vascular access device 200 is removed. Additionally or alternatively, the bottom support 900 can be configured to bend to increase the distance between adjacent end portions and enable insertion and removal of the vascular access device 200.

When implanting the vascular access device 200, the hub 202 can be implanted beneath the patient's skin, and the catheter 220 can be inserted into a targeted cardiovascular location, such as a targeted blood vessel. The blood vessel, for example, can be an artery or a vein, such as the internal jugular vein or the subclavian vein. In operation, a clinician inserts a needle (e.g., a non-coring or Huber-type needle) through the skin, through the self-sealing septum 212, and into the fluid reservoir 210. To introduce fluid (e.g., medication, contrast agent, saline solution, etc.) into the patient's blood vessel, the clinician may inject the fluid through the needle N, which then flows through the reservoir 210, the catheter 220, and into the vessel. In some cases, the physician may inject fluid through the needle to fill the reservoir 210 for postponed delivery into the vessel. To remove fluid from the vessel (e.g., to aspirate blood from the vessel for testing), the clinician can apply suction via the needle, thereby withdrawing fluid (e.g., blood) from the vessel into the catheter 220, into the fluid reservoir 210, and into the needle. After aspiration or fluid delivery, the device 200 can be flushed and/or locked. A flushing fluid (e.g., saline solution, heparin solution, etc.) can be injected through the needle, into the reservoir 210, and out of the catheter 220 such that the flushing fluid transports undesired material (e.g., residual medication, debris, etc.) out of the device 200. In some embodiments, the device 200 can be locked by delivering a volume of a locking fluid (e.g., a solution carrying at least one of sodium chloride, an anticoagulant agent, a thrombolytic agent, an antimicrobial agent, an antiseptic agent, or another suitable agent) to the catheter 220 such that the locking fluid remains in the catheter lumen and prevents or limits ingress of blood into the catheter lumen or occlusion of the catheter lumen. When the procedure is completed, the clinician removes the needle, the self-sealing septum 212 resumes a closed configuration, and the device 200 may remain in place beneath the patient's skin.

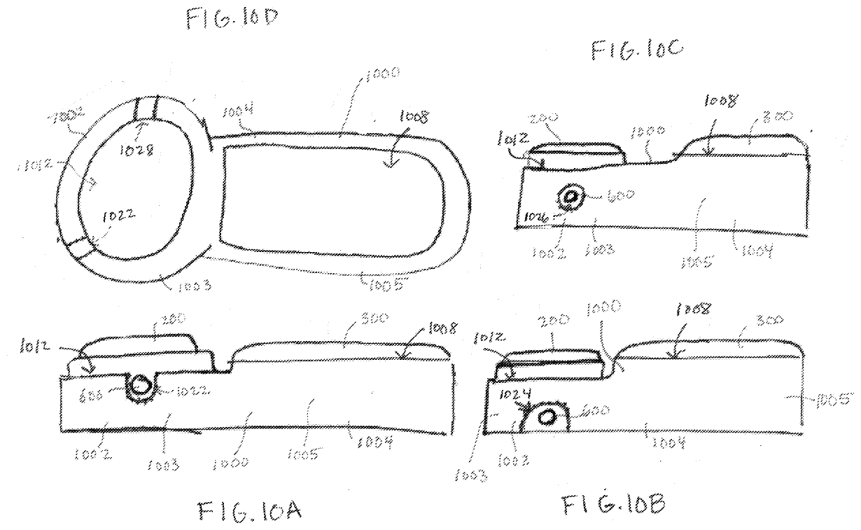

FIGS. 10A-10D illustrate several variations on a coupler 1000 comprising one or more recesses and/or openings in the sidewall 1003 of the first receiving portion 1002 to accommodate the catheter 600 of the vascular access device 200. As shown in FIG. 10A, in some embodiments the coupler 1000 comprises a recess 1022 extending downwardly from a top side 1003a of the sidewall 1003 of the first receiving portion 1002. Aside from the gap created by the recess 1022, the sidewall 1003 can be configured to extend fully around a boundary of the vascular access device 200. To secure the device 200 to the first receiving portion 1002, a user can insert the device 200 downwardly through a top side of the opening 412 until the catheter 600 sits within a portion of the sidewall 1003 defining the recess 1022. In some embodiments, the device 200 can first be positioned in the first receiving portion 1002 without the catheter 600, and then the catheter 600 can be coupled to the device 200.

As shown in FIG. 10B, in some embodiments the coupler 1000 comprises a recess 1024 extending upwardly from a bottom side 1003b of the sidewall 1003 of the first receiving portion 1002. Aside from the gap created by the recess 1024, the sidewall 1003 can be configured to extend fully around a boundary of the vascular access device 200. To secure the device 200 to the first receiving portion 1002, a user can insert the device 200 upwardly through a bottom side of the opening 412 until the catheter 600 sits within a portion of the sidewall 1003 defining the recess 1022. In some embodiments, the device 200 can first be positioned in the first receiving portion 1002 without the catheter 600, and then the catheter 600 can be coupled to the device 200.

As shown in FIG. 10C, in some embodiments the coupler 1000 comprises an opening 1026 extending through the sidewall 1003 of the receiving portion 1002 at a location between the top and bottom sides of the sidewall. Aside from the gap created by the opening 1026, the sidewall 1003 can be configured to extend fully around a boundary of the vascular access device 200. To secure the device 200 to the first receiving portion 1002, a user can guide the catheter 600 through the opening 1026 as the device 200 is inserted into the opening 412 (through the top or bottom side). In some embodiments, the device 200 can first be positioned in the first receiving portion 1002 without the catheter 600, and then the catheter 600 can be coupled to the device 200.

In some embodiments, the coupler 1000 can have more than one top or bottom recess (e.g., three recesses, four recesses, etc.). For example, as shown in FIG. 10D, the coupler 1000 can have a first recess 1022 and a second recess 1028, each at a different location along a boundary of the first receiving portion 402. This way, the first receiving portion 402 can receive the vascular access device 200 in different orientations and/or can receive a vascular access device with multiple catheters extending therefrom. Similarly, the coupler 1000 can have more than one opening (not shown). In some embodiments, the coupler 1000 has one or more top recesses 1022, one or more bottom recesses 1024, and/or one or more openings 1026 (e.g., the coupler 1000 can have one top recess 1022 and two bottom recesses 1024; a top recess 1022, a bottom recess 1024, and an opening 1026; three bottom recesses 1024 and two openings 1026, etc.). Moreover, in any of the foregoing embodiments, the coupler 1000 can comprise an elastic or otherwise deformable material that can stretch to receive the vascular access device 200 and/or electronic device 300 therein and secures the respective devices via tension. In these and other embodiments the coupler 1000 can be configured to engage the vascular access device 200 and/or electronic device 300 in a snap fit arrangement.

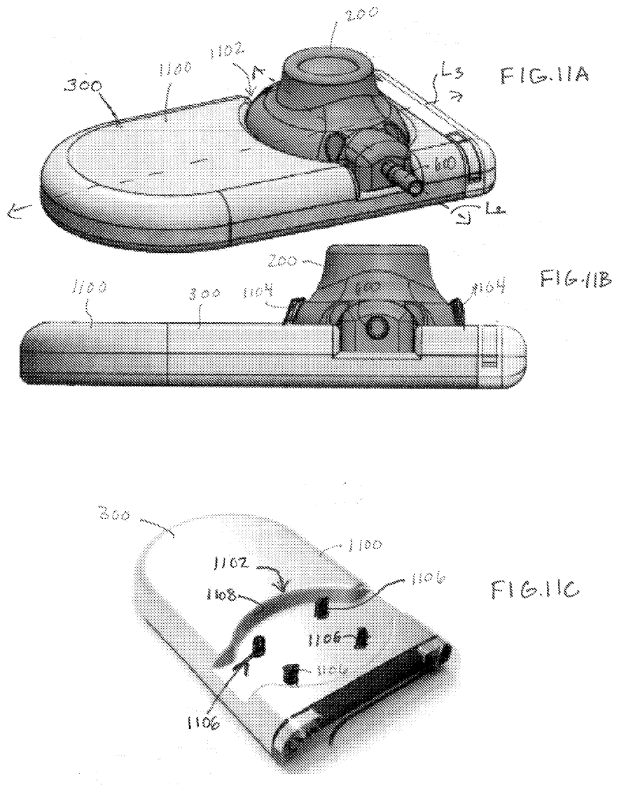

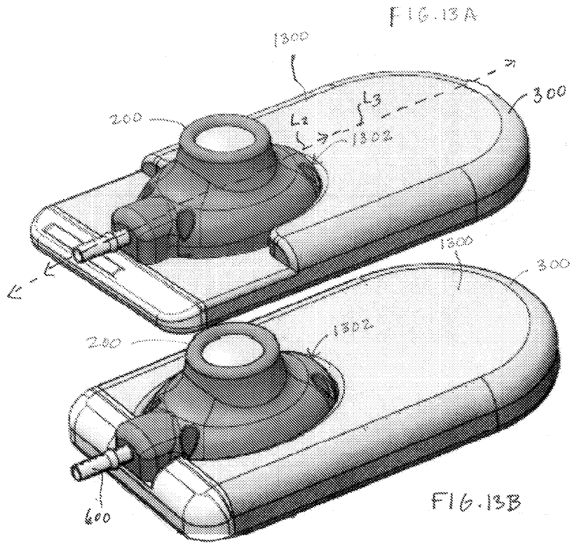

According to various aspects of the technology, the coupler may be integrated with the housing of the electronic device 300. For example, as shown in FIGS. 11A-11C, the electronic device 300 can comprise a housing 1100 (such as an hermetically sealed enclosure) that defines a recessed portion 1102 configured to receive at least a portion of the vascular access device 200. The recessed portion 1102 can have a shape corresponding to a footprint of all or some of the vascular access device 200 such that the vascular access device 200 is configured to be secured to the housing 1100 by a friction and/or snap fit arrangement. As shown in FIG. 11A, the recessed portion 1102 can be oriented such that, when the vascular access device 200 is received within the recessed portion 1102, a longitudinal axis L2 of the vascular access device 200 is angled with respect to the longitudinal axis L3 of the electronic device 300 and/or housing 1100. In some embodiments, the recessed portion 1102 can be oriented such that, when the vascular access device 200 is received within the recessed portion 1102, the longitudinal axis L2 of the vascular access device 200 is substantially perpendicular to the longitudinal axis L3 of the electronic device 300 and/or housing 1100. In certain embodiments, the recessed portion 1102 can be oriented such that, when the vascular access device 200 is received within the recessed portion 1102, the longitudinal axis L2 of the vascular access device 200 is substantially aligned with and/or parallel to the longitudinal axis L3 of the electronic device 300 and/or housing 1100 (for example, as shown in FIGS. 13A and 13B).

According to several aspects of the technology, the housing 1100 may include one or more engagement members configured to further engage and secure the vascular access device 200 while the vascular access device 200 is positioned in and/or on the recessed portion 1102. For example, as shown in FIG. 11B, the housing 1100 can include one or more protrusions 1104 configured to engage a portion of the vascular access device 200 that is not positioned within the recessed portion 1102. The protrusion(s) 1104 can surround all or a portion of the vascular access device 200. In some embodiments, for example as shown in FIG. 11C, the housing 1100 can include one or more posts 1106 positioned within the recessed portion 1101 and configured to engage a bottom surface of the vascular access device 200. The vascular access device 200 can have one or more recesses configured to receive the posts 1106. In some embodiments, the posts 1106 can be configured to be received within suture holes of the vascular access device 200.

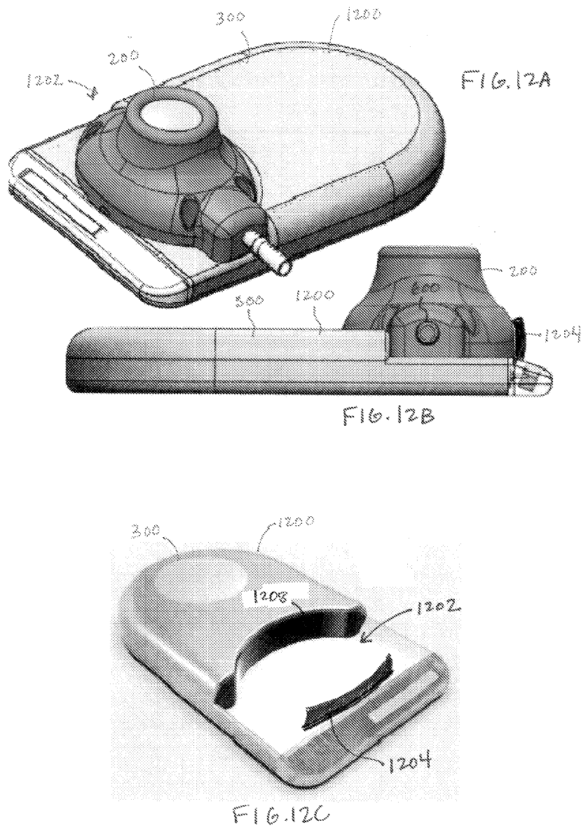

FIG. 12A shows an electronic device 300 having a housing 1200 similar to housing 1100, except in FIG. 12A, only one side of the recessed portion 1202 is vertically bound by a sidewall of the housing 1200. In several of such embodiments, the housing 1200 can include one or more engagement members to secure the vascular access device 200 to the housing 1200. The engagement members can include a laterally extending protrusion 1204 (shown in FIGS. 12B and 12C), one or more posts (not shown), and/or other features.

FIG. 13A shows an electronic device 300 having a housing 1300 similar to housing 1200, except in FIG. 13A, the recessed portion 1302 is oriented such that, when the vascular access device 200 is received within the recessed portion 1302, the longitudinal axis L2 of the vascular access device 200 is substantially aligned with and/or parallel to the longitudinal axis L3 of the electronic device 300 and/or housing 1300. FIG. 13B shows a variation of the housing 1300 in which the portion of the housing 1300 forming the vertical sidewalls that define the recessed portion 1302 are configured to surround all but the catheter 600 (and adjacent supporting structure) when the vascular access device 200 is positioned in the recessed portion 1302. In FIG. 13A, the vertical sidewalls surround only a portion of the perimeter of the vascular access device 200. The embodiments shown and described with respect to FIGS. 13A and 13B can include one or more engagement members described herein.

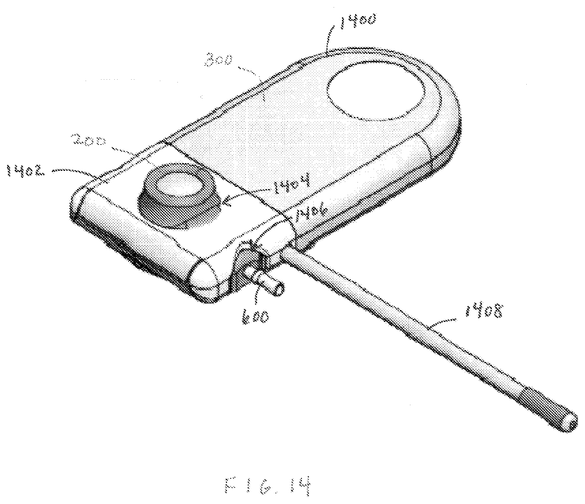

FIG. 14 shows an electronic device 300 having a housing 1400 comprising one or more openings through which a portion of the vascular access device 200 may protrude. For example, the housing 1400 can include a first opening 1404 configured to receive a top portion of the reservoir and/or septum therethrough, and a second opening 1406 configured to receive the catheter 600 therethrough. In some embodiments the housing 1400 comprises a single opening or more than two openings (e.g., three openings, four openings, etc.). The remainder of the housing 1400 can be surround and constrain the vascular access device 200. In some embodiments, the electronic device 300 optionally includes an extension 1408 comprising an electrode. While in FIG. 14 the openings 1404, 1406 are disposed proximate an end portion of the electronic device 300, in some embodiments the openings can be disposed at an intermediate portion of the electronic device 300. For example, FIGS. 15A and 15B show an electronic device 300 having a housing 1500 with first and second openings 1502, 1504 disposed at an intermediate region (e.g., away from the longitudinal ends) of the electronic device 300 with end portions 300a, 300b of the housing 1500 on either side.

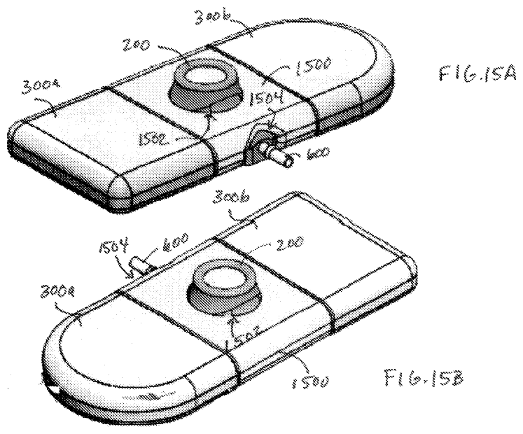

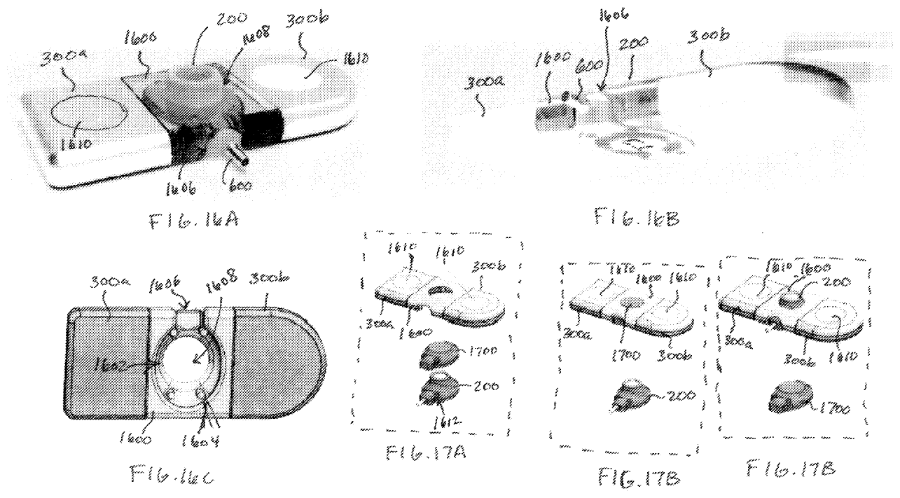

FIGS. 16A-16C show a housing 1600 similar to housing 1500. The housing 1600 has a recessed portion 1602 (best shown in FIG. 16C) configured to receive the vascular access device 200 therein. The housing 1600 can have one or more openings configured to receive one or more portions of the vascular access device 200 therethrough. For example, as shown in FIGS. 16A and 16B, the housing 1600 can include a side opening 1606 extending therethrough that is continuous with the recessed portion 1602 and configured to receive the catheter 600 (and/or adjacent supporting structure) therethrough. Additionally or alternatively, the housing 1600 can include a top opening 1608 extending therethrough that is continuous with the recessed portion 1602 a portion of the vascular access device 200, such as a top portion of the reservoir and/or septum, therethrough. In some embodiments, the housing 1600 includes one or more engagement members configured to further secure the vascular access device 200 to the housing 1600. For example, as best shown in FIG. 16C, in some embodiments a surface of the housing 1600 that defines the recessed portion 1602 can include one or more posts 1604 (only one labeled) extending therefrom and configured to mate with recesses 1612 (only one labeled in FIG. 17A) at a top portion of the vascular access device 200. In addition to or instead of the posts 1604, the housing 1600 can include other engagement members. In some embodiments, the housing 1600 can optionally include an electrode 1610 disposed on one or both sides of the recessed portion 1602.

In some embodiments, the system can further comprise a plug that is configured to be disposed within the recessed portion and/or openings in the housing of the electronic device 300 when the vascular access device 200 is not present. An example of a plug 1700 and its use is depicted in FIGS. 17A-17C. In some embodiments, the electronic device 300 may be implanted without the vascular access device 200 and without the plug 1700. In some embodiments, the electronic device 300 may be implanted only with the plug 1700. In some embodiments, the electronic device 300 may be implanted with the vascular access device 200 coupled thereto, and the vascular access device 200 can be removed at a later date and replaced by the plug 1700.

FIG. 18 shows an electronic device 300 comprising a housing 1800 that is similar to the housing 1100 shown in FIG. 11A, except in FIG. 18, the recessed portion 1802 is positioned at an intermediate portion of the housing 1800. The embodiments shown and described with respect to FIG. 18 can include one or more engagement members described herein.

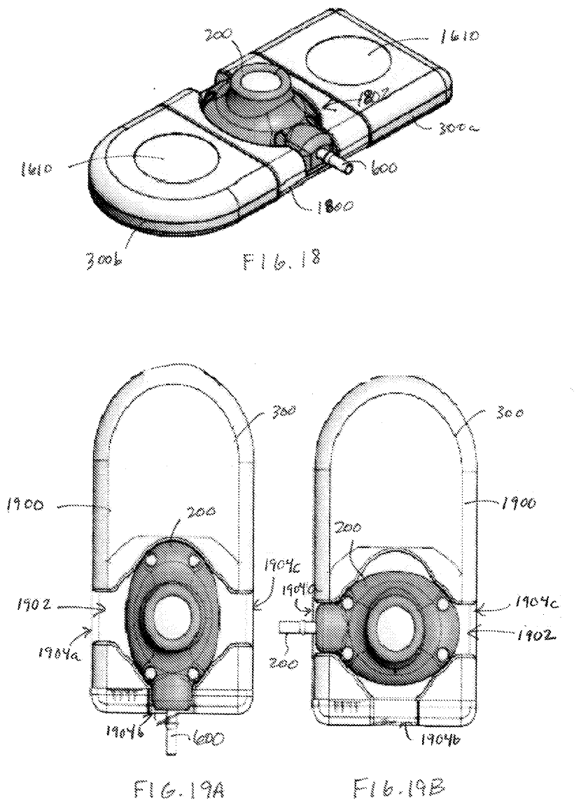

FIGS. 19A and 19B show an electronic device 300 comprising a housing 1900 that is similar to the housing 1100 shown in FIG. 11A, except in FIGS. 19A and 19B, the housing 1900 includes multiple openings 1904a, 1904b, and 1904c, each configured to receive the catheter 600 (and/or adjacent supporting structure) therethrough. The recessed portion 1902 is further shaped to accommodate the vascular access device 200 in different orientations, such as with a longitudinal axis of the vascular access device 200 substantially aligned with and/or parallel to the longitudinal axis of the housing 1900 (as shown in FIG. 19A), with the longitudinal axis of the vascular access device 200 substantially perpendicular to the longitudinal axis of the housing 1900, and/or other orientations. The shape of the recessed portion 1902 allows the operator to place the vascular access device 200 in more than one possible configuration in a subcutaneous pocket based on a patient's unique anatomy and body habitus. The embodiments shown and described with respect to FIGS. 19A and 19B can include one or more engagement members described herein.

### CONCLUSION

Although many of the embodiments are described above with respect to vascular access devices, the technology is applicable to other applications and/or other approaches, such as other types of implantable medical devices (e.g., pacemakers, implantable cardioverter/defibrillators (ICD), deep brain stimulators, insulin pumps, infusion ports, orthopedic devices, and monitoring devices such as pulmonary artery pressure monitors). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-19B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

For the purposes of this specification and appended claims, unless otherwise indicated, all numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

1. An implantable vascular access system, comprising:
a vascular access device comprising:
a reservoir body defining an interior chamber configured to receive fluid therein;
a self-sealing septum covering the interior chamber;
an outlet port in fluid communication with the chamber;
a catheter configured to be mated to the outlet port of the vascular access device;
an electronic device including one or more sensing elements configured to obtain physiological measurements; and
a coupler configured to at least partially engage the vascular access device and the electronic device, the coupler comprising:
a flexible housing having a first receiving portion configured to receive the vascular access device, a second receiving portion configured to receive the electronic device, an intermediate portion between the first receiving portion and the second receiving portion.

2. The implantable vascular access system of claim 1, wherein the intermediate portion between the first receiving portion and the second receiving portion has a flexibility configured to facilitate bending of the intermediate portion.

3. The implantable vascular access system of claim 1, wherein the intermediate portion is defined by a rigid material configured such that a fixed spacing is maintained between the first receiving portion and the second receiving portion.

4. The implantable vascular access system of claim 1, wherein the first receiving portion is defined by a sidewall and the sidewall includes at least one gap for receiving at least a portion of the catheter.

5. The implantable vascular access system of claim 4, wherein the at least one gap in the sidewall of the first receiving portion is arranged laterally opposite the intermediate portion of the coupler.

6. The implantable vascular access system of claim 4, wherein the second receiving portion is defined by a first longitudinal axis and wherein the at least one gap in the sidewall is arranged such that the catheter extends along a second longitudinal axis, such that an angle is defined between the first longitudinal axis and the second longitudinal axis.

7. The implantable vascular access system of claim 6, wherein the angle is approximately 90 degrees.

8. The implantable vascular access system of claim 6, wherein the angle is approximately 135 degrees.

9. The implantable vascular access system of claim 1, wherein the second receiving portion includes a sidewall that defines an opening complimentary in shape to the electronic device.

10. The implantable vascular access system of claim 1, wherein the first receiving portion is defined by a sidewall and a lip extends radially inwardly from a top surface of the sidewall.

11. A method for remotely monitoring the health of a patient, the method comprising:
implanting an electronic device; and
implanting a vascular access device after implanting the electronic device; and
after implanting the vascular access device, securing the vascular access device and the electronic device to a coupler, the coupler defined by a flexible housing having at least a first receiving portion for receiving the vascular access device and a second receiving portion for receiving the electronic device.

12. The method of claim 11, wherein the vascular access drive includes:
a hub comprising a housing defining a reservoir configured to receive a fluid therein; and
a catheter comprising a proximal end portion configured to be secured to the hub, a distal end portion configured to be positioned within a body lumen of a patient, and a lumen extending therethrough, the lumen being a fluid communication with the reservoir.

13. The method of claim 11, wherein the electronic device comprises:
a sensing element, the sensing element being configured to obtain data characterizing a physiological parameter of a patient.

14. The method of claim 13, wherein the electronic device comprises:
a data communications module communicatively coupled to the sensing element and configured to transmit the data obtained by the sensing element to an external computing device.

15. The method of claim 12, wherein the coupler further includes an intermediate portion positioned directly between the first receiving portion and the second receiving portion.

16. The method of claim 15, wherein the intermediate portion is defined by a flexible material.

17. A method for remotely monitoring the health of a patient, the method comprising:
implanting an electronic device; and
implanting a vascular access device after implanting the electronic device; and
after implanting the vascular access device, securing the vascular access device and the electronic device to a coupler, the coupler defined by a flexible housing having at least a first receiving portion for receiving the vascular access device and a second receiving portion for receiving the electronic device.

18. The method of claim 17, wherein the coupler further includes an intermediate portion arranged between the first receiving portion and the second receiving portion, the first receiving portion and the second receiving portion arranged laterally adjacent one another.

19. The method of claim 17, wherein the vascular access device comprises:
a hub comprising a housing defining a reservoir configured to receive a fluid therein; and
a catheter comprising a proximal end portion configured to be secured to the hub, a distal end portion configured to be positioned within a body lumen of a patient, and a lumen extending therethrough, the lumen being in fluid communication with the reservoir.

20. The method of claim 17, wherein the electronic device comprises:
a sensing element, the sensing element being configured to obtain data characterizing a physiological parameter of a patient; and
a data communications module communicatively coupled to the sensing element and configured to transmit the data obtained by the sensing element to an external computing device.
